(19) United States
(12) Patent Application Publication
(10) Pub. No.: US 2025/0160993 A1
(43) Pub. Date: May 22, 2025

(54) PROCESSING AND DISPLAY OF TISSUE TENSION

(71) Applicant: Cilag GmbH International, Zug (CH)

(72) Inventors: Frederick E. Shelton, IV, Hillsboro, OH (US); Jason L. Harris, Lebanon, OH (US); Shane R. Adams, Lebanon, OH (US)

(21) Appl. No.: 18/810,119

(22) Filed: Aug. 20, 2024

Related U.S. Application Data
(60) Provisional application No. 63/602,040, filed on Nov. 22, 2023.
(60) Provisional application No. 63/602,028, filed on Nov. 22, 2023.
(60) Provisional application No. 63/601,998, filed on Nov. 22, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.

(51) Int. Cl. A61B 90/00; A61B 17/00; A61B 17/072; A61B 34/00; G16H 20/40

(52) U.S. Cl. CPC A61B 90/06; A61B 17/07207; A61B 34/25; G16H 20/40; A61B 2017/00221; A61B 2017/07271; A61B 2017/07285; A61B 2090/064; A61B 2562/0261

(57) ABSTRACT

Systems, methods, and instrumentalities are disclosed for displaying a magnitude and location of a resulting tissue tension from an applied force to an instrument and a resulting tissue reaction. A surgical stapling device for monitoring and adapting to tissue tension during a surgical stapling procedure may measure strain on at least one component of the surgical stapling device resultant from one or more of an opposed force applied by a user and a force resisted by tissue associated with the tissue tension. The device may determine a location of the tissue tension based on historical surgical data, the opposed force, and the resisted force. The device may highlight the location of the tissue tension to aid in real-time surgical decision-making.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023;
Provisional U.S. Patent Application No. 63/602,028, filed Nov. 22, 2023;
Provisional U.S. Patent Application No. 63/601,998, filed Nov. 22, 2023,
Provisional U.S. Patent Application No. 63/602,003, filed Nov. 22, 2023,
Provisional U.S. Patent Application No. 63/602,006, filed Nov. 22, 2023,
Provisional U.S. Patent Application No. 63/602,011, filed Nov. 22, 2023,
Provisional U.S. Patent Application No. 63/602,013, filed Nov. 22, 2023,
Provisional U.S. Patent Application No. 63/602,037, filed Nov. 22, 2023, and
Provisional U.S. Patent Application No. 63/602,007, filed Nov. 22, 2023.

### BACKGROUND

Intelligent systems may be designed to affect surgical outcomes. Real-time monitoring and control may be used in staple-tissue interactions, such as managing tissue tension, strain, and stress during surgical procedures. The complexities may be compounded by variable tissue thickness and pressure differentials in staple cartridges and anvils. Real-time sensing, data analysis, and decision-making may aid in managing tissue interactions and improving patient outcomes.

### SUMMARY

A device (e.g., a powered staple) may monitor the strain or stress of a component of the staple due to an opposed force applied by the user and a corresponding force resisted by a tissue body. A relationship of tissue tension may direct a user and/or the powered stapler the location of the tension source or impact.

The tension may be macro tension affecting anatomic strictures or micro tension affecting local tissue loading. The tension may be relative to the knife motion and the resulting cut line length.

The device may measure aspects (e.g., macro tissue tension relative to the body's attachment (using a strain gauge)) and micro tissue tension within the jaws (e.g., via clamping load). The measurements may be made manually with electrical input (e.g., minimal electrical input). Instruments and/or devices configured for such implementations may include circular or endo cutters and offer more mechanical implementation.

Examples may include control parameters influencing desirable tissue outcomes. The control parameters may include tissue tension, micro tension, causes of local tissue compressive loading inconsistency, and tissue motion and distortion. The surgical stapler may fire staples progressively or simultaneously, with a possibility of pre-firing compression of the tissue. The surgical staples may exert residual forces on the tissue, leading to potential tissue strain and stress between staples. The variability in staple firing and its resulting impact may cause inconsistencies in the staple line operationally, impacting the healing properties of the tissue along its length.

Examples relate to a staple cartridge and/or anvil pocket, which may cause a pressure differential. Cartridges may have variable tissue thickness lateral to the deck of the cartridge and/or anvil varying the opening between them. The staple pockets and anvil pockets may allow tissue to expand into the pockets during the pre-firing clamp phase, leading to an irregularity in compression. Cartridges or anvils may have protrusions or recesses that allow better retention of the tissue during clamping and firing.

The proposed system may provide solutions to the effects of macro tension on tissue healing. Macro tissue tension may affect (e.g., restrict) artery and veins, leading to insufficient blood supply to a healing area or ischemia and tissue death. The indication to the health care professional (HCP) (e.g., surgeon) of the magnitude of the tissue tension applied by the stapler and/or the resulting residual tissue tension may enable the HCP to adjust accordingly.

Tension on tissue may be detected and determined (e.g., through harmonic impedance transient to predict seal quality, sensor on the shaft of the blade for loading, strain gauge, and clamp force in real time). The complete capture of tissue in the jaw staple pattern may be detected to determine full capture by using a dome sensor, pressure sensor, or continuity sensor distal to the staple pattern (e.g., on the cartridge and/or the anvil).

### BRIEF DESCRIPTION OF DRAWINGS

FIG. 1 is a block diagram of a computer-implemented surgical system.

FIG. 2 shows an example surgical system in a surgical operating room.

FIG. 3 illustrates an example surgical hub paired with various systems.

FIG. 4 shows an example situationally aware surgical system.

FIG. 5 shows an example surgical instrument.

FIG. 6 illustrates an example system diagram of determining tissue tension.

FIG. 7 illustrates an example articulating mechanism of a surgical stapler.

FIG. 8 illustrates an example architecture diagram of a smart surgical system.

FIG. 9 illustrates an example block diagram of detecting and addressing tissue tension.

FIG. 10 illustrates an example block diagram of a surgery progression using tissue tension monitoring and tissue detection.

FIG. 11 illustrates an example of a dynamic tissue interaction monitoring data set.

FIG. 12 illustrates an example block diagram of tissue detection and tissue tension monitoring.

### DETAILED DESCRIPTION

A more detailed understanding may be had from the following description, given by way of example in conjunction with the accompanying drawings.

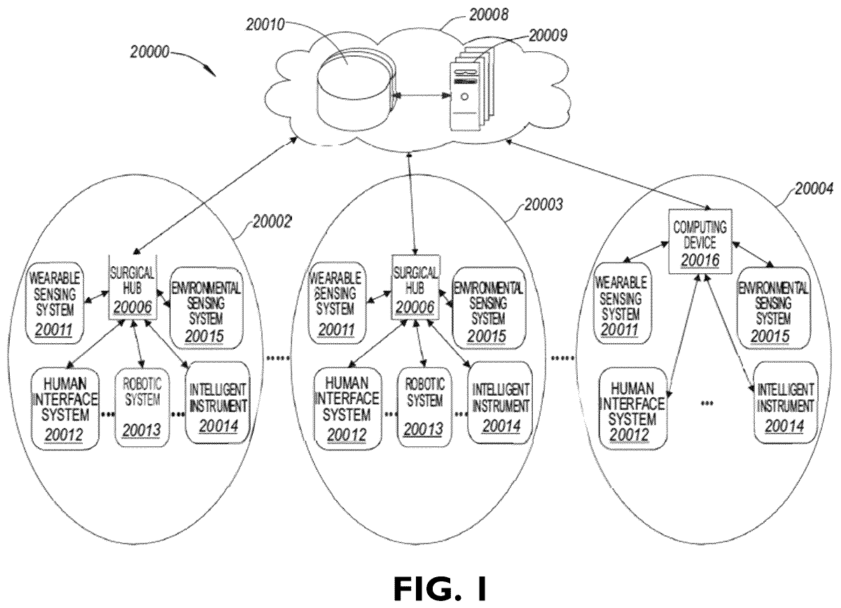

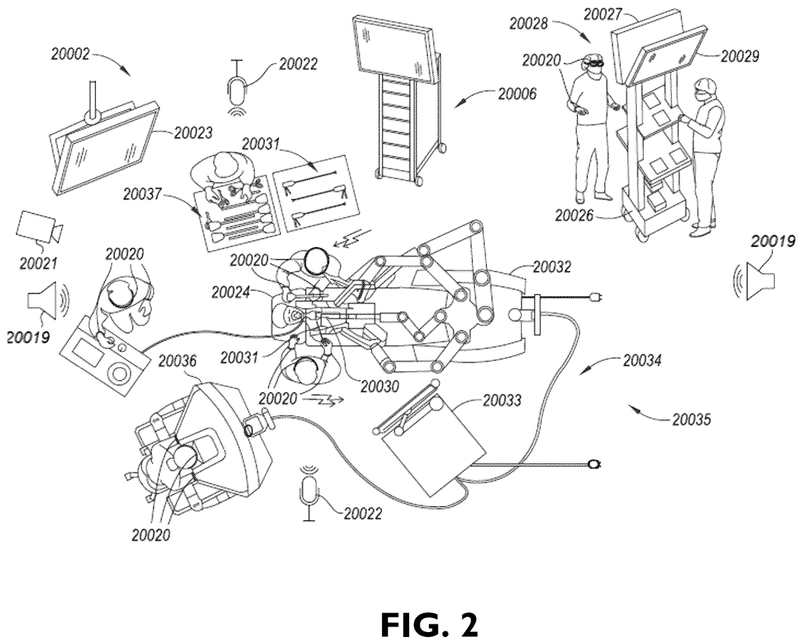

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiogramoencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

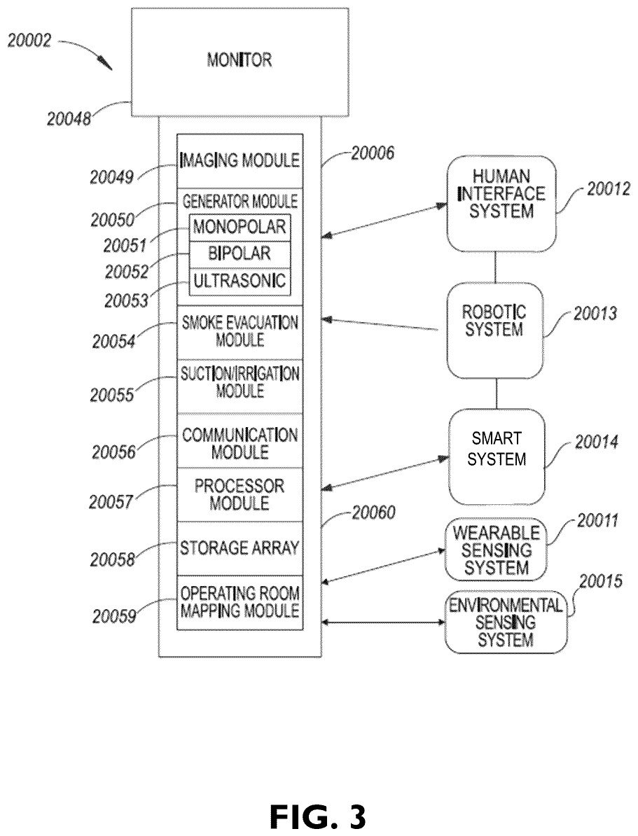

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to a non-contact sensor module. The non-contact sensor module may measure the dimensions of the operating theater and generate a map of the surgical theater using ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

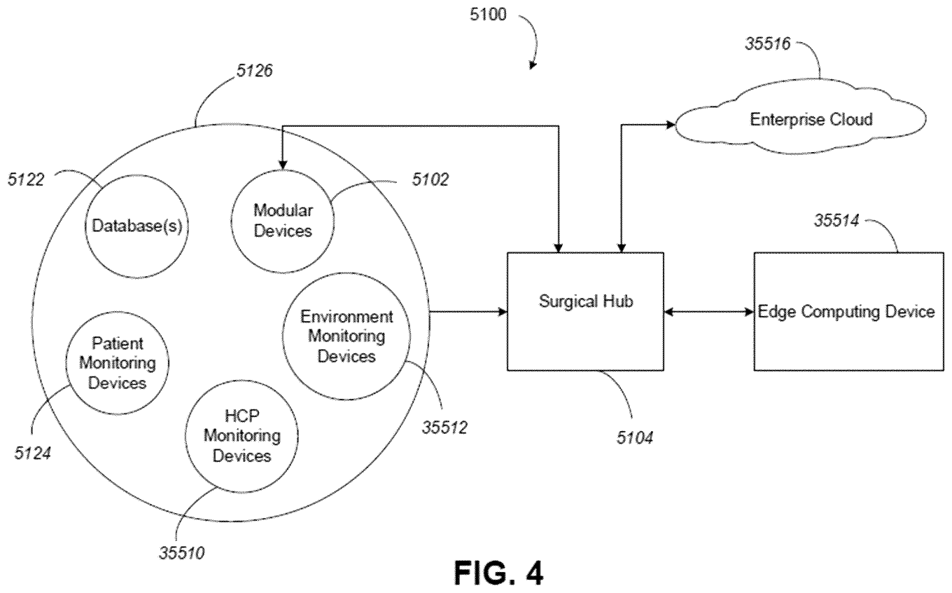

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiogramonitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient monitoring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s)) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

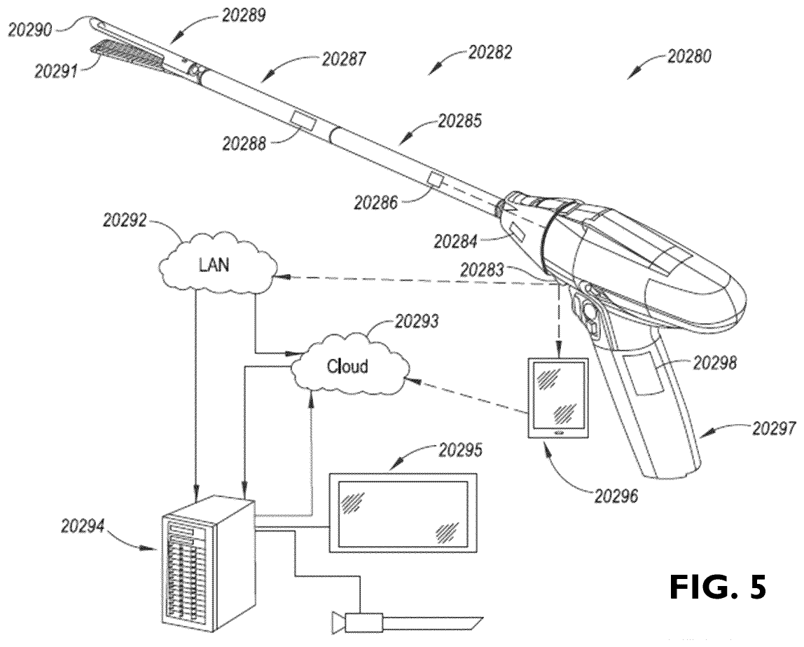

FIG. 5 illustrates an example surgical system 20280 that may include a surgical instrument 20282. The surgical instrument 20282 can be in communication with a console 20294 and/or a portable device 20296 through a local area network 20292 and/or a cloud network 20293 via a wired and/or wireless connection. The console 20294 and the portable device 20296 may be any suitable computing device. Surgical instrument 20282 may include a handle 20297, an adapter 20285, and a loading unit 20287. The adapter 20285 releasably couples to the handle 20297 and the loading unit 20287 releasably couples to the adapter 20285 such that the adapter 20285 transmits a force from a drive shaft to the loading unit 20287. The adapter 20285 or the loading unit 20287 may include a force gauge (not explicitly shown) disposed therein to measure a force exerted on the loading unit 20287. The loading unit 20287 may include an end effector 20289 having a first jaw 20291 and a second jaw 20290. The loading unit 20287 may be an in-situ loaded or multi-firing loading unit (MFLU) that allows a clinician to fire a plurality of fasteners multiple times without requiring the loading unit 20287 to be removed from a surgical site to reload the loading unit 20287.

The first and second jaws 20291, 20290 may be configured to clamp tissue therebetween, fire fasteners through the clamped tissue, and sever the clamped tissue. The first jaw 20291 may be configured to fire at least one fastener a plurality of times or may be configured to include a replaceable multi-fire fastener cartridge including a plurality of fasteners (e.g., staples, clips, etc.) that may be fired more than one time prior to being replaced. The second jaw 20290 may include an anvil that deforms or otherwise secures the fasteners, as the fasteners are ejected from the multi-fire fastener cartridge.

The handle 20297 may include a motor that is coupled to the drive shaft to affect rotation of the drive shaft. The handle 20297 may include a control interface to selectively activate the motor. The control interface may include buttons, switches, levers, sliders, touchscreens, and any other suitable input mechanisms or user interfaces, which can be engaged by a clinician to activate the motor.

The control interface of the handle 20297 may be in communication with a controller 20298 of the handle 20297 to selectively activate the motor to affect rotation of the drive shafts. The controller 20298 may be disposed within the handle 20297 and may be configured to receive input from the control interface and adapter data from the adapter 20285 or loading unit data from the loading unit 20287. The controller 20298 may analyze the input from the control interface and the data received from the adapter 20285 and/or loading unit 20287 to selectively activate the motor. The handle 20297 may also include a display that is viewable by a clinician during use of the handle 20297. The display may be configured to display portions of the adapter or loading unit data before, during, or after firing of the instrument 20282.

The adapter 20285 may include an adapter identification device 20284 disposed therein and the loading unit 20287 may include a loading unit identification device 20288 disposed therein. The adapter identification device 20284 may be in communication with the controller 20298, and the loading unit identification device 20288 may be in communication with the controller 20298. It will be appreciated that the loading unit identification device 20288 may be in communication with the adapter identification device 20284, which relays or passes communication from the loading unit identification device 20288 to the controller 20298.

The adapter 20285 may also include a plurality of sensors 20286 (one shown) disposed thereabout to detect various conditions of the adapter 20285 or of the environment (e.g., if the adapter 20285 is connected to a loading unit, if the adapter 20285 is connected to a handle, if the drive shafts are rotating, the torque of the drive shafts, the strain of the drive shafts, the temperature within the adapter 20285, a number of firings of the adapter 20285, a peak force of the adapter 20285 during firing, a total amount of force applied to the adapter 20285, a peak retraction force of the adapter 20285, a number of pauses of the adapter 20285 during firing, etc.). The plurality of sensors 20286 may provide an input to the adapter identification device 20284 in the form of data signals. The data signals of the plurality of sensors 20286 may be stored within or be used to update the adapter data stored within the adapter identification device 20284. The data signals of the plurality of sensors 20286 may be analog or digital. The plurality of sensors 20286 may include a force gauge to measure a force exerted on the loading unit 20287 during firing.

The handle 20297 and the adapter 20285 can be configured to interconnect the adapter identification device 20284 and the loading unit identification device 20288 with the controller 20298 via an electrical interface. The electrical interface may be a direct electrical interface (i.e., include electrical contacts that engage one another to transmit energy and signals therebetween). Additionally, or alternatively, the electrical interface may be a non-contact electrical interface to wirelessly transmit energy and signals therebetween (e.g., inductively transfer). It is also contemplated that the adapter identification device 20284 and the controller 20298 may be in wireless communication with one another via a wireless connection separate from the electrical interface.

The handle 20297 may include a transceiver 20283 that is configured to transmit instrument data from the controller 20298 to other components of the system 20280 (e.g., the LAN 20292, the cloud 20293, the console 20294, or the portable device 20296). The controller 20298 may also transmit instrument data and/or measurement data associated with one or more sensors 20286 to a surgical hub. The transceiver 20283 may receive data (e.g., cartridge data, loading unit data, adapter data, or other notifications) from the surgical hub 20270. The transceiver 20283 may receive data (e.g., cartridge data, loading unit data, or adapter data) from the other components of the system 20280. For example, the controller 20298 may transmit instrument data including a serial number of an attached adapter (e.g., adapter 20285) attached to the handle 20297, a serial number of a loading unit (e.g., loading unit 20287) attached to the adapter 20285, and a serial number of a multi-fire fastener cartridge loaded into the loading unit to the console 20294. Thereafter, the console 20294 may transmit data (e.g., cartridge data, loading unit data, or adapter data) associated with the attached cartridge, loading unit, and adapter, respectively, back to the controller 20298. The controller 20298 can display messages on the local instrument display or transmit the message, via transceiver 20283, to the console 20294 or the portable device 20296 to display the message on the display 20295 or portable device screen, respectively.

An example smart surgical system may include technical interactions between the health care professional (HCP), patient, surgical stapler (e.g., a linear surgical stapler or a circular surgical stapler), smart device display, and data center. The HCP may use the stapler to interface with the patient during a surgical procedure. The surgical stapler may interface with tissue and adjust operations in real-time. Data from the surgical stapler may be displayed (e.g., visually) on a smart display, aiding in immediate decision-making and highlighting tissues and tissue tension sources and results. The data center may manage and analyze surgical data, while patient associated data is redacted in conformance with patient privacy regulations (e.g., Health Insurance Portability and Accountability Act (HIPAA) rules, General Data Protection Regulation (GDPR), etc.) Patient data may be included with functionality of the surgical stapler, influencing the stapler's operations while maintaining data confidentiality standards, particularly regarding traceable patient-related information. The smart surgical system may include real-time monitoring and AI/ML algorithms within the stapler.

A surgeon as described herein may include and/or may be described by roles, capabilities, and interactions beyond an individual performing a surgery (e.g., to encompass the surgical team and a variety of extensions and devices that may be used in a surgical procedure). The surgeon may navigate scenarios and make decisions in real-time. The surgical team may include nurses, anesthesiologists, surgical assistants, HCPs, etc., and the surgeon may be used interchangeably as described herein for any member of the surgical team. In terms of technological augmentation, tools, such as robotic surgical systems, endoscopes, smart surgical staplers, and navigation systems, may modify the surgeon's capabilities. The devices may translate the surgeon's manipulations actions, provide insights into the patient's body, adjust operations, and guide the surgeon's decision-making. The devices, as they may be associated with the surgeon and the surgeon's respective navigation of the surgical procedure, may also be associated with an interchangeable with the surgeon. Control systems and user interfaces may be associated with and interchangeable with the surgeon and the technological devices.

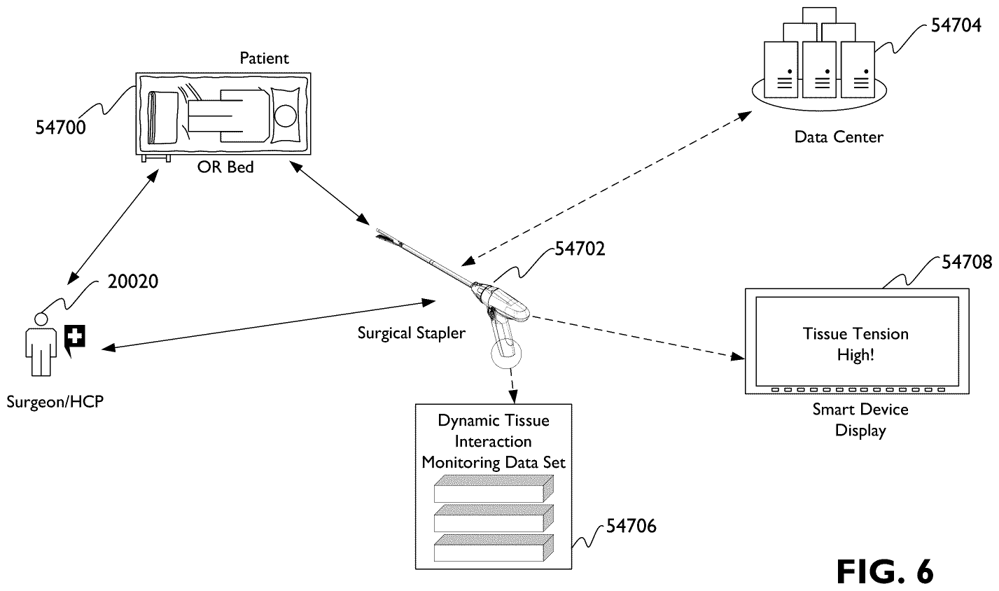

FIG. 6 illustrates an example system diagram for determining tissue tension within a surgical context, including communicative and interactive elements. The depicted patient on the operating room (OR) bed 54700 may engage in a bidirectional informational or control exchange with the HCP 20020 through instrumentation associated with the OR bed 54700. The communicative linkage may provide a continuous, dynamic interface for interactions between patient status and HCP actions.

The HCP 20020 may interface with the surgical stapler 54702 through a network for data and command exchange or via a wired connection. Although a linear surgical stapler is shown in FIG. 6 for illustration purposes, other types of surgical staplers may be used. The HCP, for example, may use the connection to control the surgical stapler, make adjustments, and receive feedback from the device, thereby modifying the surgical process by modifying the use of the stapler based on real-time data related to tissue interactions and surgical stapler performance.

Interconnectivity may exist between a data center 54704 and the surgical stapler 54702. The data center, for example, may gather, analyze, and manage surgical data associated with the surgical stapler 54702. The dynamic tissue interaction monitoring data set may include (e.g., critical) data related to tissue-stapler interactions and relevant parameters experienced during the surgical procedure.

The dynamic tissue interaction monitoring data set 54706 may be leveraged for information provided to the surgical stapler 54702. Informative and/or predictive data from the dataset may be conveyed to the display 54708, allowing practitioners (e.g., surgeons) to visualize real-time or historic data. The visualization, for example, may modify surgical decision-making by providing a visually intuitive representation of the ongoing tissue interactions during surgery.

Communication may exist between the data center 54704 and the surgical stapler 54702. A communication channel may be used to retrieve and display data from the data center 54704 and/or the surgical stapler 54702 on the smart device display 54708, as well as to receive input or directives from the display's user interface (e.g., via the surgical stapler 54702). Dynamic interaction between the components may enable data updates, synchronization, and allow the user to navigate through data sets or screens.

The surgical stapler 54702 and the smart device display 54708 may be connected, such that for example, a mutual exchange of data and commands may exist between both devices. For example, adjustments made to the stapler, or inputs through its interface, may (e.g., directly and/or indirectly) impact the data displayed, providing an adaptable interface that may facilitate a coordinated and data-informed surgical environment.

An HCP 20020 may interact with a surgical stapler 54702. The surgical stapler 54702 may provide surgical information about the patient 54700. The HCP 20020 may send inputs (e.g., control inputs) to the surgical stapler 54702. The HCP 20020 may provide control inputs or receive status indicators and/or surgical data from the surgical stapler 54702, facilitating a dynamic interaction that may adapt to evolving surgical circumstances. Operational data, feedback, or control responses may be communicated from the surgical stapler 54702 to the HCP 20020, enabling the HCP to adjust surgical tactics, techniques, or stapler operation accordingly.

Data transfer between the surgical stapler 54702 and the smart device display 54708 may be facilitated, and operational data, feedback, or control parameters pertaining to the surgical stapler may be visualized on the smart device display 54708. The HCP or other operative personnel may monitor and adjust the surgical stapler's operations in real-time based on displayed data. Control inputs or operational adjustments may be transmitted from the smart device display 54708 to the surgical stapler 54702, offering a means through which stapler operation may be modulated during a surgical procedure.

The data center 54704 may interact with the surgical stapler 54702 to process, transmit, or receive data relevant to tissue-stapler interactions. Data from the dynamic tissue interaction monitoring data set 54706 (e.g., inclusive of prior stapling events, tissue characteristic data, or other relevant variables) may be communicated to the surgical stapler 54702 via the data center 54704. The data may inform or adapt operational parameters of the stapler during a procedure and/or after the procedure (e.g., via a software update uploaded from the data center to the surgical stapler). Data originating from the surgical stapler 54702 may be communicated to the data center 54704, contributing to the dynamic tissue interaction monitoring data set 54706 for future use, analysis, reference, or update (e.g., software update).

The surgical stapler 54702 may be configured for patient data confidentiality. The surgical stapler 54702 may be configured to process and transmit data pertinent to tissue-stapler interactions, with provisions for redacting any patient-related information within transmitted data sets. Artificial Intelligence and Machine learning (AI/ML) algorithms may analyze surgical setups and configurations, facilitating a comparison thereof. The configuration may allow for the data to be rendered non-traceable and/or fully redacted, thereby upholding privacy standards (e.g., HIPAA or GDPR privacy rules), while enabling the derivation of operational parameters for the surgical stapler 54702.

The surgical stapler 54702 may be configured for real-time monitoring and detection of tissue and/or tissue tension within the surgical device. The surgical stapler 54702, by the local configuration may operate independently of external data transmissions and may use AI/ML within the dynamic tissue interaction monitoring data set 54706 for improvement and adaptation of operational parameters (e.g., of the surgical stapler 54702).

Communication may be established between the data center 54704 and the smart device display 54708 (e.g., via the surgical stapler). The display may present data associated with the dynamic tissue interaction monitoring surgical data set 54706 (e.g., or other sources) and allow for the transmission of data or control inputs back to the data center 54704 from the surgical stapler. The interaction between the data center 54704 and the smart device display 54708 may incorporate the surgical stapler 54702 such that surgical data may be shared and operational parameters may be adjusted.

Interaction with the patient on the OR bed 54700 may influence the operation of the surgical stapler 54702, and physiological data, patient status, and relevant parameters may influence the stapler's operational parameters or be influenced by the stapler's operational parameters. For example, tissue data, patient status, or procedural progression may interact with the operational parameters of the surgical stapler 54702, adapting functionality of the surgical stapler 54702 or providing data that may inform surgical decisions or adjustments.

The surgical stapler 54702, in communication with entities such as the HCP on 20020, smart device display 54708, and data center 54704, may facilitate a communicative environment where data is exchanged and operational parameters may be adjusted. The interaction and data exchange (e.g., via a network or wired connection) may provide a means for modifying surgical outcomes, safety, and procedural efficiency through the operation of the surgical stapler 54702 and interconnected surgical tools or systems.

A surgical stapler may include an articulating mechanism, which may be associated with surgical precision and patient outcomes including different levels of tissue tension. The mechanism may include an articulation pivot 54710 around a first axis (e.g., horizontal), which may augment the stapler's maneuverability during surgical procedures. Multiple pivot points may be associated with managing tissue interactions at various different angles and strengths. The articulation point along with real-time sensing and data analysis may allow the stapler to adjust to diverse surgical environments.

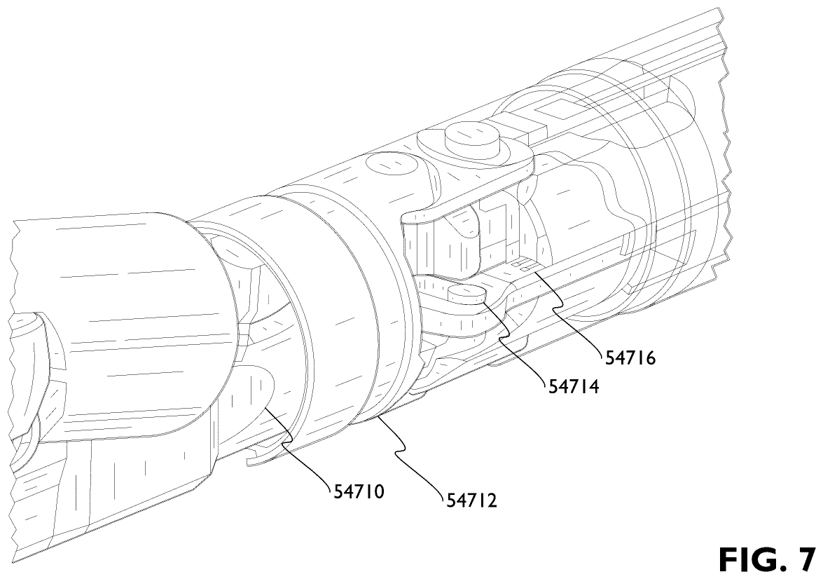

FIG. 7 illustrates an example articulating mechanism of a surgical stapler. An articulation pivot 54710 around a first Axis (e.g., horizontal axis) may be included. The pivot may be associated with the surgical stapler's maneuverability during a surgical procedure, allowing the surgical stapler to maneuver with flexibility during operations. The articulation pivot's design may manage tissue interactions, particularly in varying tissue thickness and pressure differentials. The articulation point 54710, combined with real-time sensing and data analysis may modify the stapler's ability to adapt to different surgical environments, thereby enabling precise tissue handling and staple application.

A surgical stapler shaft 54712 may be included in the surgical stapler. The shaft may be designed to transmit the forces between a user to the tissue, facilitating controlled staple deployment (e.g., through wiring or via a sensor including wireless communication. The shaft may incorporates technology to monitor and respond to tissue strain or stress caused by an opposed force applied by the user and the corresponding force resisted by the tissue body. The shaft may aid in determining the relationship of tissue tension, guiding the user and the powered stapler to identify the tension source or impact, whether it be macro tension affecting anatomic structures or micro tension influencing local tissue loading.

An articulation pivot 54714 around a second axis may be associated with an articulation pivot around a vertical axis. The articulation pivot 54714 may complement the articulation pivot 54710, providing a degree of movement that may aid in precise surgical operations. The integration of one or more pivots may aid in managing micro tissue tension within the jaws of the device, thereby contributing to consistent tissue compressive loading and reducing the variability in staple firing and its impact on the healing properties of the tissue.

A sensor 54716 may be associated with detecting tissue tension. The sensor may enable real-time monitoring of tissue tension during surgical procedures. The sensor may aids in detecting both macro and micro tissue tensions, providing feedback to the healthcare professional. The sensor may detect harmonic impedance transients to predict seal quality, measure strain gauge and clamp force in real time, and enable complete capture of the tissue in the jaw staple pattern.

An example smart surgical system may exist within an I/O device, which may include user interfaces and data connectivity ports for communication during surgical procedures. Tissue tension data may be analyzed and stapling actions may be adapted to the live tissue environment within a surgical procedure. A tissue tension application, including monitoring and detection modules, may aid in adjusting stapling mechanisms based on real-time tissue characteristics. A processor and memory within the smart surgical system, including volatile and non-volatile types may include the monitoring and detection modules, which may be accessed by the processor. Functionality may include network connectivity, enabling interaction with systems such as a data center within a HIPAA or a GDPR protected boundary, ensuring compliance with data security and privacy standards across the system's interconnected functionalities.

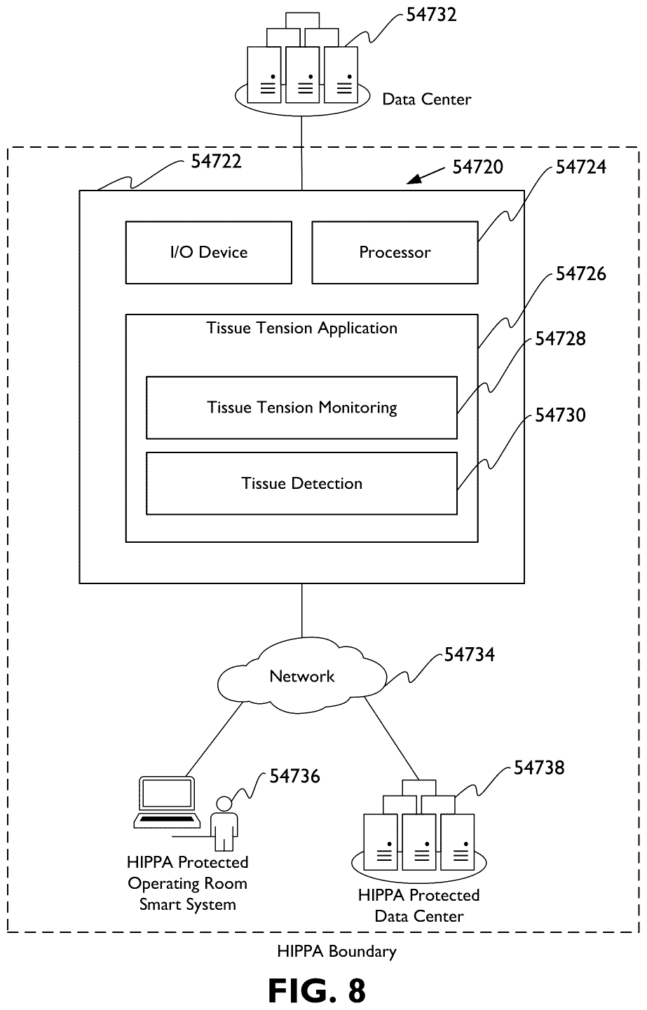

FIG. 8 illustrates an example architecture diagram 54720 of a smart surgical system. The smart surgical system may be included in an I/O device 54722, which may be integrated in the smart surgical stapler 54702. The I/O device 54722 may include interfaces such as touch panels, voice recognition modules, and numerous data connectivity ports, aiding communication between the user and the device during surgical procedures. The I/O device may provide control and communication in the shifting nature of surgical activities.

The embedded processor 54724 may manage computational tasks and adopt architectures depending on the aspects of surgical stapling operations. Examples of such processors may include multicore processors, GPUs, and ASICs, and any of the processors, GPUs, or ASICs may be selected based on their capability to handle the computational, graphical, and data processing inherent in real-time surgical situations. The processor 54724 may facilitates computations and analyze tissue data to facilitate adaptive stapling actions that are associated with live tissue environments.

The tissue tension application 54726, including the tissue tension monitoring module 54728 and tissue detection module 54730 may work in tandem to monitor, interpret, and react to the tissue tension variations during stapling. The stapling mechanisms may be adjusted to coincide with the present tissue tensions and textures, for example, to minimize tissue damage and enable stapling alignment and deployment.

Memories may be utilized within the system, such as volatile memory types like RAM for fast data retrieval and non-volatile storage types like SSDs or HDDs for persistent storage. The memory may provide data storage, access, and recovery, enabling continuity and recoverability of system operations.

The smart surgical system's functionality may extends via network 54734, enabling data and control to be exchanged with systems, such as data center 54732. The I/O device, network, and a HIPAA protected operating room smart surgical system 54736 and Health Insurance Portability and Accountability Act (HIPAA) protected data center 54738 may be within a HIPAA boundary. HIPAA may refer to HIPAA and applicable data privacy regulations, such as General Data Protection Regulation (GDPR), Payment Card Industry Data Security Standard) PCI DSS, California Consumer Privacy Act (CCPA), etc., which may be applicable at jurisdictional levels including region, country, or state. The systems may provide supplementary database capabilities, computational resources, and data storage, enabling the system's capability to expand and backup data. By interfacing with systems like the HIPAA protected operating room smart surgical system 54736 and HIPAA protected data center 54738, the system may enable data handling, storage, and processing to be compliant with data security and privacy standards that safeguard patient data among the interconnected functionalities of the system in compliance with HIPAA.

An example smart surgical system may include a user input/force application module, communicating with a tissue resistance module for adjustments during stapling procedures. A tissue tension monitoring module may update algorithms based on the input from the user and tissue resistance, influencing staple firing adjustments during staple use. The staple integrity and healing module may be associated with staple deployment and tissue healing, using data to affect (e.g., positively) post-operative outcomes. Interactions with a tissue detection module may enable adaptive monitoring based on varying tissue types, while the display feedback module may translate tension metrics to the HCP, aiding in procedural adjustments. The folded tissue detection and correction module may identify and addresses tissue misconfigurations, mitigating issues for stapling across varied tissue conditions. The folded tissue detection and correction module may communicate with the display feedback module to provide alerts and insights to the HCP.

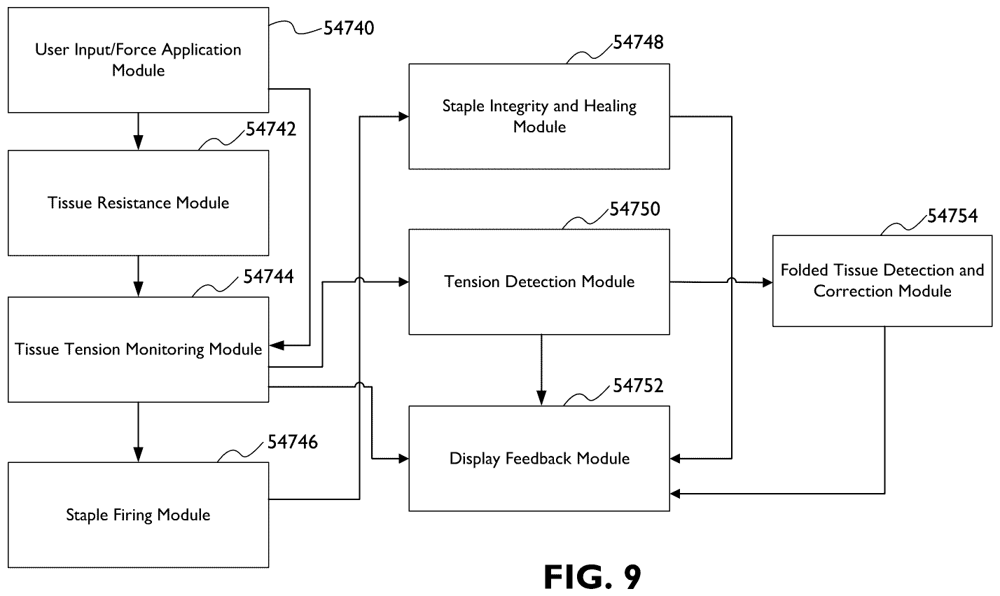

FIG. 9 illustrates an example block diagram of detecting and addressing tissue tension. The user input/force application module 54740, for example, may communicate with the tissue resistance module 54742. The user input/force application module 54740 may provide pertinent data regarding HCP inputs or detected force metrics during stapler operation, influencing or guiding algorithms or mechanisms within the tissue resistance module 54742 that are related to real-time tissue resistance assessments or adjustments.

In an interface to both the user input/force application module 54740 and the tissue resistance module 54742, the tissue tension monitoring module 54744 may process or otherwise utilize data from the user input/force application module 54740 and the tissue resistance module 54742 to develop, refine, or inform tissue tension monitoring processes or algorithms. The algorithms may adapt to tissue conditions or HCP inputs, affecting tissue tension assessments or adjustments throughout a surgical procedure.

The staple firing module 54746 may derive input from the tissue tension monitoring module 54744, facilitating, for example, adjustments or alterations to staple deployment based on monitored tissue tension metrics. The interaction may involve the modulation of staple firing parameters, such as force, speed, or timing, adapting to real-time or anticipated tissue conditions, and modifying stapling efficacy or safety.

In communication with the staple firing module 54746, the staple integrity and healing module 54748 may influence actions related to the integrity of deployed staples and associated tissue healing trajectories. Data relevant to deployed staple parameters, tissue conditions, or other relevant metrics to monitor, predict, or enhance staple integrity and tissue healing may be used throughout and following a stapling procedure.

The tissue tension monitoring module 54744 may interact with a tissue detection module 54750, enabling the incorporation of data or feedback associated with detected tissue types or conditions into tissue tension monitoring processes. In examples, algorithms or processes within the tissue tension monitoring module 54744 may adapt or refine tissue tension assessments or predictions based on data associated with detected tissue types, for example, enhancing the specificity or accuracy of tissue tension monitoring mechanisms.

Interaction between the tissue tension monitoring module 54744 and the display feedback module 54752 may facilitate the translation of tissue tension metrics or status indicators to a user interface, modifying HCP awareness or enabling data-informed adjustments to technique or stapler operation during a procedure. The incorporation of real-time or predicted tissue tension data into a visual display may affect the ability of HCPs to anticipate, identify, or address tissue tension issues proactively.

Communication between the staple integrity and healing module 54748 and the display feedback module 54752 may address aspects related to the visualization of data or feedback pertinent to staple integrity or tissue healing processes. The real-time monitoring of staple status or healing trajectories may be analyzed, enabling adjustments to surgical strategy or post-operative care based on visualized data.

In examples, the tissue detection module 54750 may facilitate data flow or feedback to the folded tissue detection and correction module 54754. The flow and/or feedback may involve the identification and correction of issues related to folded tissue during stapling processes, affecting the efficacy or safety of stapler operation in varied tissue conditions.

The folded tissue detection and correction module 54754 may engage, for example, in identifying instances where tissue folding may be present or imminent during the application of surgical staples, thereby providing a mechanism by which the tissue misconfigurations may be addressed during the surgical procedure. In examples of macro tension, involving distinct tissue structures, the module 54754 may employ sensors or algorithms to detect variations or anomalies in tissue interaction, which may signal potential or unfolding tissue folding events or anomalies, enabling preemptive or responsive alterations to stapling parameters or approaches to mitigate such events and promote staple engagement and tissue healing across the interfacing tissue structures.

Considering micro tension within a singular tissue structure, the module 54754 may leverage its sensing or computational capabilities to precisely discern localized tension disparities and may determine and communicate micro-scale adjustments to the stapling mechanics or parameters. The data analyzed by module 54754 for the operations may stem from integrated sensors or external sources, which may report tissue attributes or conditions and inform the module's management of stapling mechanics in the micro-scale tissue dynamics. The adaptive strategies implemented by the module 54754 may facilitate localized adaptations in stapling operations to manage and mitigate issues associated with micro tension disparities and tissue folding within the single tissue structure.

The folded tissue detection and correction module 54754 may modify the surgical stapling approach and mechanics in alignment with the tension within and across the engaged tissue structures. The modulations navigated by module 54754 may affect mechanical and functional outcomes of the stapling process and mitigate occurrences of tissue misconfigurations, trauma, or poor staple engagement across the macro and micro tension encountered within the surgical environment.

Interactions between the folded tissue detection and correction module 54754 and the display feedback module 54752 may include communication of data, status indicators, or alerts relevant to folded tissue issues to a user interface. By providing actionable insights or notifications related to detected and corrected folded tissue issues, the capacity for timely issue resolution or adjustment during stapling procedures may be affected.

The surgical smart system may include a user input module that may be in communication with components to affect precision during stapling procedures. The smart surgical system may include a tissue resistance module and a tissue tension monitoring system, which may include (e.g., refine) algorithms based on inputs received and influencing staple firing adjustments. Staple integrity and healing be included, and the surgical smart system may use data to positively impact post-operative outcomes. Interactions with a tissue detection module may be included, which may enable adaptive monitoring based on the varying types of tissue encountered. A display feedback module may provide and/or translate tension metrics to the HCP, which may aid in procedural adjustments. A folded tissue detection and correction module may identify and address tissue misconfigurations and mitigate issues associated to stapling across varied tissue conditions. The folded tissue detection and correction module may communicate with the display feedback module.

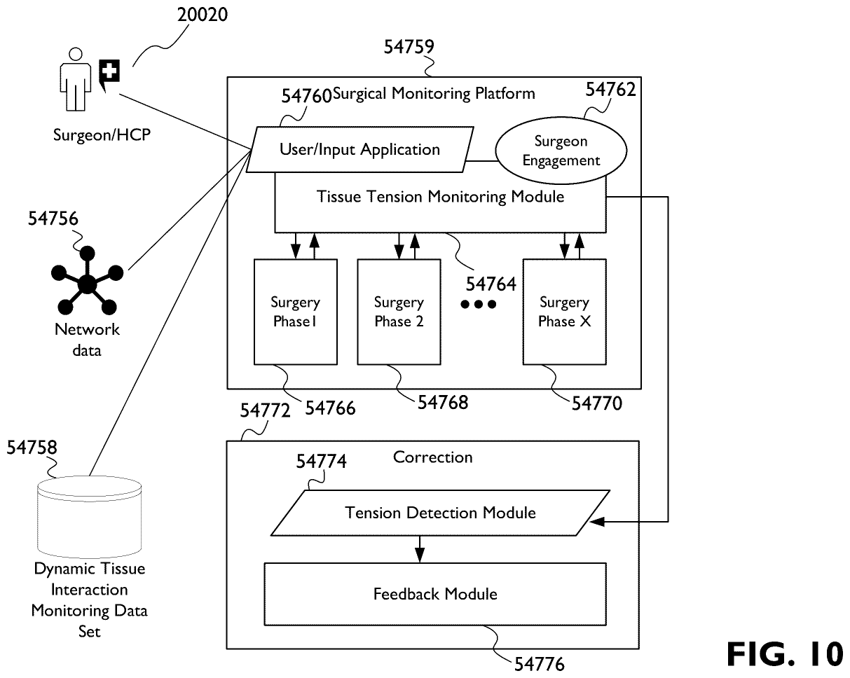

FIG. 10 illustrates an example block diagram of a surgery progression using tissue tension monitoring and tissue detection. The HCP 20020, network data 54756, and a dynamic tissue interaction monitoring data set 54758 may collectively contribute to the user input application 54760. The combined input may facilitate informed decision-making and adaptability throughout the surgical procedure by enabling real-time data flow and adjustability based on the dynamic tissue interaction.

A surgical monitoring platform 54759 may assimilate and coordinate modules for a comprehensive surgical management solution. The tissue tension monitoring module 54764 may be directly interfaced with the user/input application 54760, HCP engagement 54762, surgery phase 1 54766, surgery phase 2 54768, and surgery phase X 54770. Any of the surgical phases may be used to determine whether specific tension parameters and tissue interactions are monitored and managed, providing the HCP with data that may affect precision and safety during a surgery phase.

At correction 54722, real-time response to deviations from expected tissue tension parameters may be included. Within the correction 54722, the tension detection module 54774 may interpret variations in tissue tension and determine any discrepancies from expectations within the surgical environment. The feedback module 54776 may receive the interpreted data, enabling the provision of responses or modifications within the ongoing surgical procedure.

The communication between the tissue tension monitoring module 54764 and the tension detection module 54774 may enable a flow of data pertaining to tissue tension, which may facilitate detection and response to a variance from an expected tension parameters. Surgical interventions may be (e.g., promptly) adapted in response to the dynamic tissue interactions encountered during the procedure.

The surgical monitoring platform 54759 may utilize the data from the user input application 54760 and associated modules to holistically monitor and guide progression through the predetermined surgical phases. The surgical team may anticipate and prepare for subsequent stages of the procedure and determine that resources and considerations are aligned for a phase.

In assessing the impact on tissue during surgical procedures, the measurement and display of perceived tissue tension and its resulting effects may be considered. The surgical stapler may monitor the strain or stress on at least one of its components, which may arise from an opposing force applied by the user and/or a resisting force from the tissue body. The stapler may display the correlation between tissue tension and the applied force, highlighting the location of the tissue tension impact or source. Such tissue tension may be associated with macro tension, impacting anatomic structures, or as micro tension, affecting local tissue loading. The tensions may be observable in relation to the motion of the knife and the resulting cut line length. Examples may include the inadvertent incomplete capture of a vessel within the staple pattern. Inadvertent and incomplete capture may lead to variable tissue compression, causing the tissue to extend beyond the staple pattern onto the cartridge's nose while remaining trapped under a portion of the anvil tip (e.g., an incomplete and unintended capture of the vessel).

In examples including micro tension, local tissue movement or distortion may be minimized. Factors may contribute to inconsistencies in local tissue compressive loading, tissue motion, and distortion. The stapler's clamping and firing aspects, with the local configuration of the cartridge surface, may introduce irregularities along the length of the stapler pattern. The irregularities may affect a (e.g., one) portion of the staple line operationally, influencing the consistency, formed staple tightness, and staple form. The surgical stapler may operate using progressive firing mechanisms, releasing a set of staples after another longitudinally and/or by simultaneously firing all staples. In an example surgical stapler including progressive firing mechanisms, the presence of an I-beam moving from proximal to distal may create a running wave of force on the tissue. The force wave may be based on the progressive local closure of the device, resulting in a distal tissue flow while the staples in that area are being projected through the tissue and/or formed. Regardless of the firing mechanism, a degree of pre-firing compression of the tissue may be observed with variations in load magnitude along the jaws of the stapler. The variation may lead to overloading of tissue within the jaws, with closure loads surpassing predetermined levels, and may for example, result in an initial force detection mechanisms gauging tissue thickness relative to the tissue gap of the cartridge.

Tissue may be a viscoelastic material (e.g., behavior of tissue may be both force and time dependent on compression). Force in one region (e.g., more force in one region) under the same time may cause compression (e.g., more compression) until the tissue is allowed to stabilize, for example, taking from a few seconds for semi-incompressible tissues (e.g., vessels) to up to over 30 seconds for (e.g., highly) compressible but thick tissues (e.g., the stomach).

Heterogeneous tissue composition may be described herein. Firing staples into a compressed form of tissue, followed by releasing the tissue to recover to a more natural compression state, may cause the deployed staples to exert residual forces on the tissue. The residual force affected on the tissue may result in residual tissue strain, as the staples may be deployed in a condition that is different from a tissue state (e.g., a native tissue state). The strain and stress between the staples may be illustrated in examples including tear-drop shaped holes that do not conform to the staple leg, and/or distortion of the staple line from a linear to an arc shape. Misalignment may occur when a circular stapler is fired through a linear staple line. Overstuffing the crotch of the stapler and variations in the design of the staple cartridge and anvil pockets may cause pressure differentials and irregularities in compression. The design features may impact staple integrity and healing, leading to bleeding, inconsistent staple height or form, organ contents escape, or inconsistent remaining blood flow and healing properties of the tissue along its length. In examples, local flow may distort the path of the staple as the staple is ejected and formed, causing malformations or preventing portions of the line from forming, which may for example, result in bleeding, local tissue destruction, or loss of containment.

Maintaining perfusion within the macro tension category may include measurements and assessments for example surgical outcomes. The measurement of bending in the shaft or channel retainer along the X and Y axes may be associated with particular surgical outcomes and may be executed by instrumenting a channel retainer or closure tube with the bending measured via strain gages. The tension may be measured by pulling forces that are lateral to the articulation axis (e.g., by gauging the resultant strain in the distal articulation link.

In examples (e.g., involving a single pivot articulation system), the articulation link or locking pin may a restraining member for the head (e.g., when the end-effector is subjected to externally applied forces). The nature of the forces may be discerned by employing directionality strain gages configured in arrangements such as a half or full bridge with 45-degree or orthogonal arrays relative to each other. Signals derived from the configurations may enable the determination of forces related to lifting, lateral dragging, other actions, etc. An external smart surgical system, such as an imaging system or a non-contact tissue strain measurement system employing light imaging with discrete tissue marking or laser-based systems may be included. Use of such tools may aid in establishing the directionality of forces in relation to the orientation and location of the end-effectors.

The relative arrangement of the forces' directionality, when considered in conjunction with the end-effector's location and orientation may be included, which may aid in distinguishing between operational forces, accidentally externally applied forces, and macro tissue tension. Macro tissue tension, may have implications as macro tissue tension may restrict arteries and veins (e.g., leading to insufficient blood supply to the healing area, ischemia, and tissue death). In clinical scenarios focused on healing, HCPs may attend to residual tissue tension on organs, especially between surgical sites (e.g., new surgical sites) and existing attachment points, as well as their fixation to other anatomical locations.

HCPs may be provided with indications of the magnitude of tissue tension being applied by the stapler and/or the magnitude of the resultant residual tissue tension. HCPs may relieve tension, reposition the device, or mobilize the organ before delivering staples (e.g., a surgical event that may be irreversible).

In examples, a segmentectomy where a portion of the lower sigmoid or descending portion of the colon afflicted with cancer may require resection. Such a procedure may be associated with a re-anastomotic connection. The remainder of the descending colon may be mobilized with a portion of the transverse colon from the surrounding connective tissue, allowing for repositioning after the removal of the cancerous part for reattachment. In examples, an endocutter may be employed to cut and seal the sigmoid portion above and below the cancerous segment. If the tissue tension is displayed, the display may allow the HCP to assess whether additional mobilization may reduce tension. The residual tension may be associated with the introduction of a circular stapler. The remaining ends may be pulled together for reconnection, reforming the colon tube. A display indicating the magnitude of macro tissue tension on the circular or linear stapler may allow for repositioning, release, reclamping, and/or mobilization (e.g., additional mobilization).

Tension on tissue may be detected and determined. Harmonic impedance transients may predict seal quality, tissue tagging may be identified (which may highlight potential pad wear qualities, and sensors may be used on the shaft of the blade for loading. Sensors being used on shaft of the blade for loading may include using strain gauges on components such as the clamp arm, clamp force in real-time, shaft, handle, and the clamping drivetrain.

The time duration of clamping may be considered along with user input regarding the procedure and the specific organ involved. A setting may be introduced for an organ mode, for instance, for the liver, and confirming the target tissue or organ via video feed. The angle of the jaws relative to the vessel may be associated with the rotational angle and energy spread over a larger area (which may lead to a weaker than desired seal). Video feed may aid in finding the angle such that no tension on the tissue exists.

Precision and efficacy of surgical procedures may be affected when using vascular staplers on arteries and veins. The complete capture of tissue may occur within the jaw staple pattern. For full capture and preventing inadequate securing of tissue, the tissue's location within the jaws may be ascertained through the implementation of various sensors such as a dome sensor, pressure sensor, or continuity sensor, which may be positioned distally to the staple pattern on either the cartridge or the anvil. If the tissue extends beyond the staple pattern, the contact sensors may detect presence of the tissue and alert the user, highlighting the inadequacy of the capture within a single firing action.

Cutline length consistency may be affected. A consistent cutting behavior may mitigate the impact of cutting through a previous staple or dragging a staple (e.g., the previous staple) along the cut line. Staple line perpendicularity may be associated with crossing staple lines. Efforts may be made to produce consistent cut line lengths.

Factors contributing to inconsistencies in cutline length may include the speed of the knife, which may range between 5 mm/sec and 25 mm/sec in powered staplers and may be faster in hand-actuated staplers. The force applied to the staple systems may cause deflections. Articulation of the stapler head may be associated with the length of the firing member that is not on the device's centerline tending to splay or have a differing resulting functional length. Insufficient holding load on the tissue during firing may push the tissue (e.g., the entire tissue) and staple line out of the device's end as the device is progressively fired. Weak holding loads on thick tissue may result in a 45 mm resulting line and cut from a 60 mm stapler.

A short cutting may occur when a device is fired in a straight configuration. In a 60 mm cutline, a discrepancy may result in up to 5-10 mm less cutting. The surgical smart system may attempt to minimize such effects by calibrating the stroke of the device based on the articulation angle, monitoring the internal systems' splay, and keeping track of the applied forces.

An example smart surgical system may include a dynamic tissue interaction monitoring displayed in and a smart surgical module that may oversee interactions within the surgical workflow. A user input/force may aid in transmitting HCP inputs to the system. Tissue resistance may be measured during surgical interactions and tissue tension may be monitored for changes in tissue tension. Staple firing and deployment may be adjusted according to tissue state. Staple integrity and healing may be proactively monitored for post-application status of staples and may assess integrity and healing progression to reduce post-operative risk. Tension detection may be associated with tissue tension data and may allow for surgical adjustments. Display feedback may provide data throughout the procedure. Folded tissue detection and correction may identify and rectify tissue folds and may safeguard from inadvertent tissue folding during the procedure mitigate future complications. Individual patient parameters may use patient-specific data to modify the surgical approach.

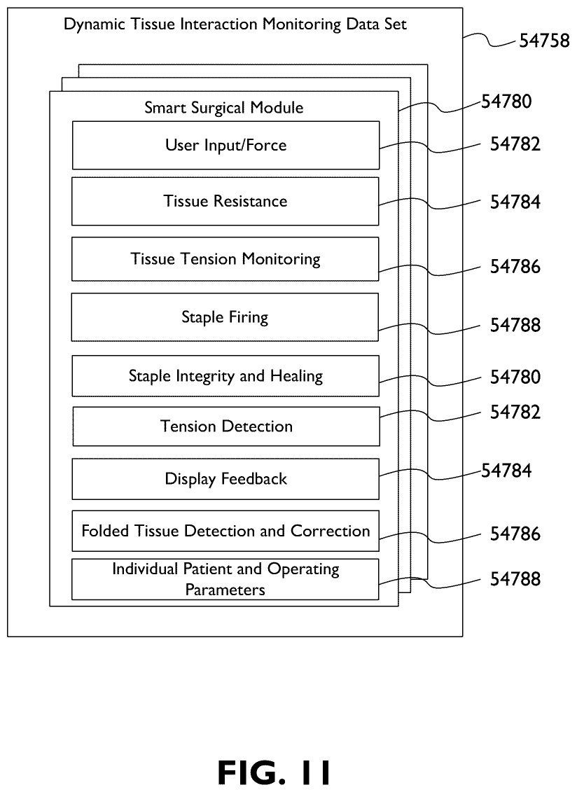

FIG. 11 illustrates an example of a dynamic tissue interaction monitoring data set 54758. The smart surgical module 54780 may form a means for managing multifaceted interactions and responses within the surgical workflow. The smart surgical module 54780 may manifest allow for the facilitating a synchronized interaction between sub-modules. A sub-module may be purposed for a function within the surgical procedure.

The user input/force 54782 may be implemented to offer an interface through which HCP inputs and forces can be effectively communicated to the system, thus facilitating a platform for user engagement and determining that HCP inputs (e.g., whether manual inputs or system directives) are processed.

The tissue resistance 54784 may function to gauge the resistance applied to and by tissues during surgical interaction. The surgical steam may receive, based on the tissue resistance 54784, data pertinent to tissue status and potential impediments or considerations that may be included into the surgical strategy.

Tissue tension monitoring 54786 may be devised to monitor and evaluate fluctuations and stable readings in tissue tension throughout the procedure. Data may be provided that may influence surgical decisions.

Staple firing 54788 may be used to regulate the deployment of staple congruence such that the surgical plan is suitable for the tissue state. Staple firing 54788 may govern the precision and reliability of staple applications, adjusting and modifying as per the specifications of the live tissue environment.

The module for staple integrity and healing 54780 may proactively monitor the post-application status of staples, determining the integrity of the staple line and determining whether the healing progression and likelihood is within acceptable and anticipated parameters. The tissue healing in a particular manner may mitigate risks related to post-operative staple failure or tissue healing (e.g., suboptimal tissue healing).

Tension detection 54782 may provide real-time data concerning tissue tension, allowing the surgical approach to be modified and adapted to manage the specifications posed by the ongoing status of the tissue environment.

Display feedback 54784, acting as a communication hub, may provide the surgical team with data and analytical insights throughout the procedure, suggesting that data-driven decisions may be facilitated and supported throughout the procedure's progression.

The module for folded tissue detection and correction 54786 may be used to identify and correct tissue folds and determine that the surgical procedure is protected against inadvertent mismanagement or misalignment of tissue while mitigating potential future complications related to tissue mismanagement.

Individual patient and operating parameters 54788 may make use of patient-specific data, determining whether the surgical procedure and technique are uniquely adapted and tailored to the specific requirements and limitations of the patient.

An example surgical smart system may be configured for tissue detection and tension monitoring in surgical scenarios (e.g., utilizing integrated sensors in a surgical stapling device to measure strain and capture the interaction dynamics between the user, device, and tissue). The system may employ historical surgical data to modify the precision of tissue tension location determination for alignment with real-time inputs and past experiences. Highlighting the determined tissue tension on the smart system's display may facilitate real-time surgical decision-making, with responses and actions for mitigate detected tension. The smart surgical system may differentiate between macro and micro tensions and provide data into tissue behavior and structural changes during surgery.

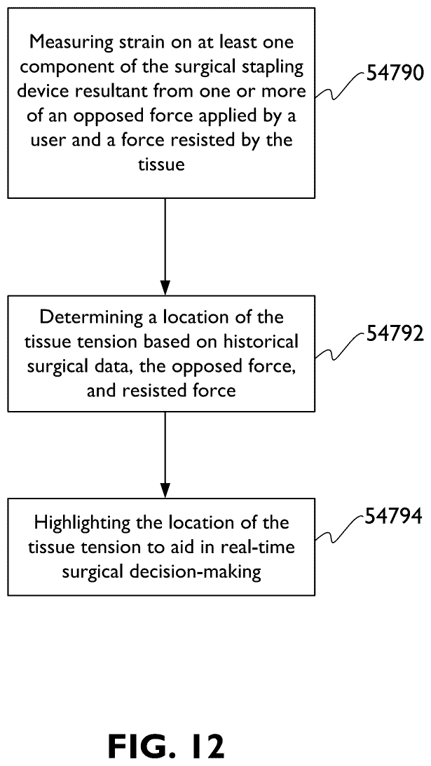

FIG. 12 illustrates an example block diagram of tissue detection and tissue tension monitoring. At 54790, strain may be measured on at least a component of a surgical stapling device, accounting for opposed force applied by a user and force resisted by the tissue, using integrated sensors within the device. The measurement of the strain, utilizing sensors embedded within the surgical stapling device, may enable the device to capture interaction dynamics between the user, the device, and the pertinent tissue.

At 54792, a determination of tissue tension location may be made (e.g., using historical surgical data including the opposed and resisted force data). The historical data (e.g., retrieved from previous surgeries on the same patient) may aid in determining the precision of tissue tension location. The data may be used to determine that present surgical activities are aligned with real-time inputs and associated with the historical data. The historical data (e.g., of one or more of the patient historical data, surgical data, and/or the like) may be retrieved from a local storage (e.g., data center) with the protected network (e.g., protected data center) of the healthcare facility or from a central location (e.g., data center that may be located outside the protected network). The data center may be a regional enterprise server or a global enterprise server.

At 54794, the determined location of the tissue tension may be highlighted to facilitate a real-time surgical decision-making process. The highlighting of the determined location of the tissue tension may be done on a display of the smart system, and an adaptive response to the tissue tension, including corrections and actions such as easing or mobilizing the tissue, may aid to lessen or eliminate the detected tension.

A difference between macro tensions affecting anatomical structures and micro tensions impacting local tissue loads may be noted by the system (e.g., providing a differently highlighted location accordingly for the micro tensions and the macro tensions. The discernment and response to the detected tension may be made based on an understanding of tissue behaviors and identifying variable tissue compression. Such tissue loading may be indicative of tissue extending beyond a staple pattern onto a nose of a stapling device cartridge, or identifying an incomplete vessel capture within the staple pattern, and clarifying a view of tissue behavior and structural changes during surgical proceedings.

1. A method for a surgical stapling device of monitoring and adapting to tissue tension during a surgical stapling procedure, the method comprising:
measuring strain on at least one component of the surgical stapling device resultant from one or more of an opposed force applied by a user and a force resisted by tissue associated with the tissue tension;
determining a location of the tissue tension based on historical surgical data, the opposed force, and the resisted force; and
highlighting the location of the tissue tension to aid in real-time surgical decision-making.

2. The method of claim 1, wherein the historical surgical data is retrieved from a previous surgery, and wherein the historical surgical data enhances the determination of the location of the tissue tension.

3. The method of claim 1, wherein the determined location of the tissue tension is associated with macro tension, and wherein the macro tension affects anatomic structures, and wherein the method further comprises:
highlighting the location of the tissue tension in relation to the macro tension affecting the anatomic structures.

4. The method of claim 1, wherein the determined location of the tissue tension is associated with micro tension, and wherein the micro tension affects a local tissue load, and wherein the method further comprises:
highlighting the location of the tissue tension in relation to the micro tension affecting the local tissue load.

5. The method of claim 4, further comprising:
determining variable tissue compression, wherein the variable tissue compression is indicative of tissue extending beyond a staple pattern onto a nose of a stapling device cartridge of the surgical stapling device while the tissue is located beneath an anvil tip portion of the surgical stapling device; and
determining an incomplete vessel capture within the staple pattern based on the variable tissue compression.

6. The method of claim 1, wherein the determination of the location of tissue tension is further based on a movement of a cutting instrument and a resulting cut line length from the movement.

7. The method of claim 1, wherein the strain is measured using sensors integrated within the surgical stapling device.

8. The method of claim 1, wherein the method further comprises:
correcting the tissue tension based on the determined location of the tissue tension, wherein the correction comprises any of easing the tissue tension or mobilizing the tissue, and wherein the correction eliminates the tissue tension.

9. A surgical stapling device for monitoring and adapting to tissue tension during a surgical stapling procedure, the device comprising a processor, wherein the processor is configured to:
measure strain on at least one component of the surgical stapling device resultant from one or more of an opposed force applied by a user and a force resisted by tissue associated with the tissue tension;
determine a location of the tissue tension based on historical surgical data, the opposed force, and the resisted force; and
highlight the location of the tissue tension to aid in real-time surgical decision-making.

10. The device of claim 9, wherein the historical surgical data is retrieved from a previous surgery, and wherein the historical surgical data enhances the determination of the location of the tissue tension.

11. The device of claim 9, wherein the determined location of the tissue tension is associated with macro tension, and wherein the macro tension affects anatomic structures, and wherein the processor is further configured to:
highlight the location of the tissue tension in relation to the macro tension affecting the anatomic structures.

12. The device of claim 9, wherein the determined location of the tissue tension is associated with micro tension, and wherein the micro tension affects a local tissue load, and wherein the processor is further configured to:
highlight the location of the tissue tension in relation to the micro tension affecting the local tissue load.

13. The device of claim 12, wherein the processor is further configured to:
determine variable tissue compression, wherein the variable tissue compression is indicative of tissue extending beyond a staple pattern onto a nose of a stapling device cartridge of the surgical stapling device while the tissue is located beneath an anvil tip portion of the surgical stapling device; and
determine an incomplete vessel capture within the staple pattern based on the variable tissue compression.

14. The device of claim 9, wherein the determination of the location of tissue tension is further based on a movement of a cutting instrument and a resulting cut line length from the movement.

15. The device of claim 9, wherein the strain is measured using sensors integrated within the surgical stapling device.

16. The device of claim 9, wherein the processor is further configured to:
modify the surgical stapling procedure based on the highlighted tissue tension location, wherein the modification comprises decreasing a potential tissue damage risk from macro or micro tensions.

17. A device for monitoring and adapting to tissue tension during a surgical stapling procedure, the device comprising a processor, wherein the processor is configured to:
measure strain on at least one component of the surgical stapling device resultant from a force applied during the surgical stapling procedure;
determine a location of the tissue tension based on historical surgical data and the force applied during the surgical stapling procedure; and
highlight the location of the tissue tension to aid in real-time surgical decision-making.

18. The device of claim 17, wherein the historical surgical data is retrieved from a previous surgery, and wherein the historical surgical data enhances the determination of the location of the tissue tension.

19. The device of claim 17, wherein the determined location of the tissue tension is associated with macro tension, and wherein the macro tension affects anatomic structures, and wherein the processor is further configured to:
highlight the location of the tissue tension in relation to the macro tension affecting the anatomic structures.

20. The device of claim 17, wherein the determined location of the tissue tension is associated with micro tension, and wherein the micro tension affects a local tissue load, and wherein the processor is further configured to:
highlight the location of the tissue tension in relation to the micro tension affecting the local tissue load.
